(19) United States
(12) Patent Application Publication
(10) Pub. No.: US 2019/0110774 A1
(43) Pub. Date: Apr. 18, 2019

(54) WEARABLE HEALTH-MONITORING DEVICES AND METHODS OF MAKING AND USING THE SAME

(71) Applicant: Infrasonix Inc., Lawrenceville, GA (US)

(72) Inventor: Nigel J. Flynn, Flowery Branch, GA (US)

(21) Appl. No.: 16/164,404

(22) Filed: Oct. 18, 2018

Related U.S. Application Data
(60) Provisional application No. 62/573,851, filed on Oct. 18, 2017.

(51) Int. Cl. A61B 8/00; A61B 7/04; A61B 8/08

(52) U.S. Cl. CPC A61B 8/4227; A61B 7/04; A61B 8/4411; A61B 8/46; A61B 8/5223; A61B 8/565; A61B 8/4494

(57) ABSTRACT

Wearable health-monitoring devices that are suitable for detecting one or more signals/sounds produced by an animal's body, such as a human body, are disclosed. Methods of making and using wearable health-monitoring devices are also disclosed.

### CROSS-REFERENCE TO RELATED APPLICATIONS

This patent application claims the benefit of priority to U.S. Provisional Patent Application Ser. No. 62/573,851 filed on Oct. 18, 2017 and entitled “WEARABLE HEALTH-MONITORING DEVICES AND METHODS OF MAKING AND USING THE SAME,” the subject matter of which is hereby incorporated by reference in its entirety.

### FIELD OF THE INVENTION

The present invention is directed to wearable health-monitoring devices. The present invention is further directed to methods of making and using wearable health-monitoring devices.

### BACKGROUND

In the United States each year, more than 600,000 people die from cardiovascular disease accounting for one in four mortalities. Of these mortalities, statistics show that some 325,000 people experience their first heart attack and die within approximately one hour of this event due to cardiac arrest. Worldwide these numbers are twenty times larger.

Undiagnosed heart disease is a leading cause of death because no cost-effective technology exists in the hands of every front-line medical professional to identify subjects with either early onset or advanced cardiovascular disease states during an annual check-up. This diagnosis would allow the patient to receive early preventative counseling and recommendations of life-style changes, or in more advanced cases, referral to a cardiologist for timely follow-up care.

Since 1817 medical professionals have relied upon a stethoscope to provide acoustic diagnostic information for medical decisions. The stethoscope continues to provide useful information, but it suffers some limitations. The detection device that the stethoscope relies upon is the human ear, which has the ability to hear sound in a frequency range from approximately 20 to 20,000 hertz. Unfortunately, sound within this frequency range is extensively absorbed by human cellular tissue, which makes it difficult to detect or identify the original source of a given sound.

Recently, a significant breakthrough in sensor technology has allowed scientists for the first time to be able to accurately detect and record sound at frequencies well below the 20 hertz threshold of human hearing, namely “infrasound.” Such a breakthrough in sensor technology was the development of sensors as disclosed in U.S. Pat. No. 8,401,217, the subject matter of which is hereby incorporated by reference in its entirety. Another breakthrough in sensor technology was the development of sensors used in infrasonic stethoscopes as disclosed in U.S. Patent Application Publication Number 2016/0095571, now U.S. Pat. No. 9,445,779, the subject matter of which is hereby incorporated by reference in its entirety.

As evidenced by recent developments in sensor technology, medical diagnostic technology has developed significantly over the last 50 years. Medical diagnostic technology developed over the last 50 years includes, but is not limited to, anatomical methods (such as X-ray Computed Tomography, Computed Tomography Coronary Calcium Score, intima-media thickness [IMT], and intravascular ultrasound [IVUS]), as well as physiological methods (such as lipoprotein analysis, HbA1c, Hs-CRP, and homocysteine), all of which have profoundly influenced both the detection of the onset and treatment of cardiovascular disease. Unfortunately, cardiovascular disease rates and the dramatic mortality rates associated with them have only increased during the last 50-year period.

The anatomical methods directly measure some aspects of the actual process of atherosclerosis itself and therefore offer the possibility of early diagnosis, but these methods are very expensive, involve significant radiation dosages, as in the example of X-ray Computed Tomography (100-1,000 times higher than conventional X-rays—even 5,000 times in the case of multiple uses), or are significantly invasive, as in the case of intravascular ultrasound. The physiological methods are less expensive, but they are not able to quantify the disease state or directly track disease progression. More importantly, existing medical diagnostic technology is unable to achieve the mass proactive monitoring of cardiovascular health that is necessary in order to significantly impact the world's cardiovascular health.

The power of primary proactive monitoring of cardiovascular health is not only that it allows individuals to bring about proactive improvements to their own cardiovascular health through the guidance and assistance of their primary care physicians, but it also permits at-risk patients to be quickly identified, so that they can receive the necessary follow-up attention from a consultant cardiologist, instead of becoming a tragic statistic of mortality from the fourth leading cause of death. This referral will allow a cardiologist to conduct additional follow-up diagnostic work which may include some of the methods outlined earlier, knowing that the high cost and radiation exposure is justified in the context of a defined pre-existing condition, which appropriately justify their use.

For the reasons provided above, efforts continue to further develop medical technology, devices and procedures to further battle cardiovascular disease and other diseases.

### SUMMARY

The present invention addresses some of the difficulties and problems discussed above by the discovery of two solutions, one a ‘medical technology’ designed to be operated by medical practitioners in a general practitioners office, hospital or care facility to evaluate patients during a patient evaluation, the other a ‘consumer product’ designed to be worn by an end user to continuously monitor their health.

Medical Product

Medical Practitioners require a ubiquitous technology capable of being deployed in a General Practitioners office, Hospitals or Care Facilities which can quickly and simply report the cardiovascular health of every patient during a short medical examination. The report will immediately indicate the extent of any disease state through a simple numeric output with a score from 1-100 throughout their life. The score will allow the patient to instantly grasp their health state, and enable practitioners to provide proactive guidance to help them improve their cardiovascular health. The technology will also allow medical practitioners to quickly identify patients who require immediate referral to a cardiologist and follow-up care. The technology will additionally provide detailed information concerning conditions of the human heart. The technology is sufficiently advanced to enable it to detect all sixty-four conditions of the human heart and immediately report the presence of any of these conditions.

Consumer Product

A consumer product designed to be worn as a watch or similar wearable device (e.g., clothing, etc.). The wrist worn product will reside in a position on the wrist that allows it to obtain health information similar to that described for the medical product. The wrist worn product will have the ability to diagnose all sixty-four conditions of the human heart.

The consumer product is designed to provide direct feedback to the wearer if an adverse health information is detected by the device. An example of this would be: “Contact your general practitioner for follow-up” or “Call 911 now”. The consumer product will also communicate patient infrasound data directly to their general practitioner via the internet or other healthcare professional for their immediate review. The healthcare professional will possess software that will allow them to quickly diagnose a disease state based upon the information provided from the patient's watch and enable them to suggest a treatment approach.

The success of the consumer product will also rely upon a communication ecosystem which offers the secure transmission of health data from the wearer to the institutions and medical professionals responsible for the care of the individual.

The wrist worn product or similar wearable device (e.g., clothing, etc.) will also have the ability to provide a cardiovascular disease score from 1-100 to enable the patient to understand their cardiovascular health. This will be achieved by linking the wrist worn sensor or similar wearable device (e.g., clothing, etc.) with a second sensor (i.e., a second sensor positioned along or within a patient that can measure other patient health properties, such as a sensor that detects and processes an EKG output). The information gathered will be evaluated and scored with using cloud based software or an application.

The Technology

The technology is based upon a newly developed sensor, discussed above and herein, that is capable of detecting sound below the limit of human hearing in a frequency range from 0.01 to 20 Hertz known as ‘Infrasound’. Infrasound emerging from the human body is a rich source of medical information concerning the heart and cardiovascular system as well as a variety of other disease states.

The product developed from this technology will provide detection and diagnosis based on infrasound emerging from the human body. The technology does not introduce any energy into the body. The first product will focus upon conditions of the heart and cardiovascular system. The technology also requires the simultaneous collection of EKG information to enable the precise time-related correlation infrasound information from the heart.

The medical product utilizes a sensor within a circular housing which is approximately 1.5 inches in diameter. The consumer product will detect information using a smaller sensor, which is rectangular in shape with a length of approximately 1 inch and a width of approximately ¼ inch to ½ inch. The sensor dimensions mirror that of placing two fingers upon the surface of the skin in the area of the wrist adjacent to the upper palm of the hand where an artery is close to the surface of the skin and easily monitored.

The Outcome

The cumulative outcome of the technology enables subjects to understand their heart and cardiovascular disease state from early in life and be able to proactively manage their heart and cardiovascular health throughout their lives with expert help and guidance from their general practitioner.

The aim of the technology is to prevent the incidence of heart attacks and strokes related to poor cardiovascular health by ensuring that every subject is able to control their cardiovascular health through the information which the device provides and with guidance from their general practitioner concerning proactive steps that they can take to improve their heart health.

Accordingly, the present invention is directed to a consumer product in the form of wearable health-monitoring devices. In one exemplary embodiment, the wearable health-monitoring device of the present invention comprises: (1) a substrate that is attachable to a patient's body; and (2) one or more sensors attached to or embedded within the substrate, wherein each of the one or more sensors comprises: a body comprising a proximal end, a distal end, a body side wall extending between the proximal end and the distal end, an end wall at the proximal end, and an aperture at the distal end; a body coupler attached to the distal end and over the aperture so as to form a substantially air-tight seal, wherein the body coupler is capable of engagement with the patient; a cavity surrounded by the body side wall, the end wall and the body coupler; a conductive backplate within the cavity and defining a backchamber between the conductive backplate and the end wall; a conductive membrane within the cavity, the conductive backplate and the conductive membrane being spaced apart from each other to form a capacitor; and a preamplifier board in electrical connection with the conductive backplate, the preamplifier (i) being capable of measuring a capacitance between the conductive membrane and the conductive backplate and converting the measured capacitance into a voltage signal, and (ii) being parallel to each of the conductive backplate and the conductive membrane, each of said one or more sensors being capable of detecting acoustic signals in a frequency range of 0.01 Hertz (Hz) to 30 Hz (or any value between 0.01 Hz and 30 Hz including end points 0.01 Hz and 30 Hz, in increments of 0.01 Hz, e.g., 0.05 Hz, or any range of value between 0.01 Hz and 30 Hz including end points 0.01 Hz and 30 Hz, in increments of 0.01 Hz, e.g., from 0.81 Hz to 8.75 Hz).

The present invention even further relates to methods of making wearable health-monitoring devices. In one exemplary embodiment, the method of making a wearable health-monitoring device comprises: attaching or embedding one or more sensors onto or in a substrate that is attachable to a patient's body, each of the one or more sensors comprising: a body comprising a proximal end, a distal end, a body side wall extending between the proximal end and the distal end, an end wall at the proximal end, and an aperture at the distal end; a body coupler attached to the distal end and over the aperture so as to form a substantially air-tight seal, wherein the body coupler is capable of engagement with the patient; a cavity surrounded by the body side wall, the end wall and the body coupler; a conductive backplate within the cavity and defining a backchamber between the conductive backplate and the end wall; a conductive membrane within the cavity, the conductive backplate and the conductive membrane being spaced apart from each other to form a capacitor; and a preamplifier board in electrical connection with the conductive backplate, the preamplifier (i) being capable of measuring a capacitance between the conductive membrane and the conductive backplate and converting the measured capacitance into a voltage signal, and (ii) being parallel to each of the conductive backplate and the conductive membrane, each of said one or more sensors being capable of detecting acoustic signals in a frequency range of 0.01 Hz to 30 Hz.

The present invention even further relates to methods of using wearable health-monitoring devices. In one exemplary embodiment, the method of using a wearable health-monitoring device comprises positioning the herein-described wearable health-monitoring device so that one or more sensors of the wearable health-monitoring device can detect sound from one or more locations within the patient's body, each of the one or more sensors comprising: a body comprising a proximal end, a distal end, a body side wall extending between the proximal end and the distal end, an end wall at the proximal end, and an aperture at the distal end; a body coupler attached to the distal end and over the aperture so as to form a substantially air-tight seal, wherein the body coupler is capable of engagement with the patient; a cavity surrounded by the body side wall, the end wall and the body coupler; a conductive backplate within the cavity and defining a backchamber between the conductive backplate and the end wall; a conductive membrane within the cavity, the conductive backplate and the conductive membrane being spaced apart from each other to form a capacitor; and a preamplifier board in electrical connection with the conductive backplate, the preamplifier (i) being capable of measuring a capacitance between the conductive membrane and the conductive backplate and converting the measured capacitance into a voltage signal, and (ii) being parallel to each of the conductive backplate and the conductive membrane, each of said one or more sensors being capable of detecting acoustic signals in a frequency range of 0.01 Hz to 30 Hz.

These and other features and advantages of the present invention will become apparent after a review of the following detailed description of the disclosed embodiments and the appended claims.

### BRIEF DESCRIPTION OF THE FIGURES

The present invention is further described with reference to the appended figures, wherein:

FIG. 1 depicts a view of an exemplary wearable health-monitoring device of the present invention;

FIG. 2 depicts a view of the exemplary wearable health-monitoring device shown in FIG. 1 on the wrist of a patient;

FIG. 3 is a cross-sectional view of an exemplary sensor suitable for use in the exemplary wearable health-monitoring device shown in FIGS. 1-2;

FIG. 4 is a flow diagram of electronics suitable for use with the exemplary wearable health-monitoring device shown in FIGS. 1-2 so as to process signals from the sensor used in the exemplary wearable health-monitoring device of the present invention; and

FIG. 5 is a flow diagram of electronics/software suitable for use with the exemplary wearable health-monitoring device shown in FIGS. 1-2 so as to process signals from the sensor and generate three dimensional images for display.

### DETAILED DESCRIPTION

To promote an understanding of the principles of the present invention, descriptions of specific embodiments of the invention follow and specific language is used to describe the specific embodiments. It will nevertheless be understood that no limitation of the scope of the invention is intended by the use of specific language. Alterations, further modifications, and such further applications of the principles of the present invention discussed are contemplated as would normally occur to one ordinarily skilled in the art to which the invention pertains.

The present invention is directed to wearable health-monitoring devices. The present invention is further directed to methods of making wearable health-monitoring devices. The present invention is even further directed to methods of using wearable health-monitoring devices.

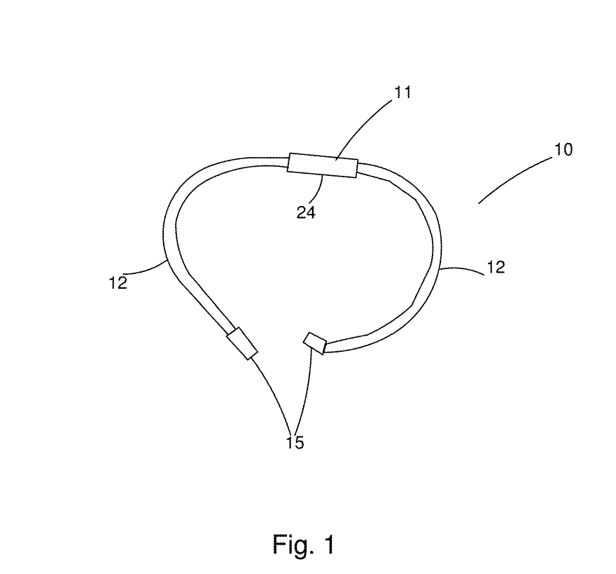

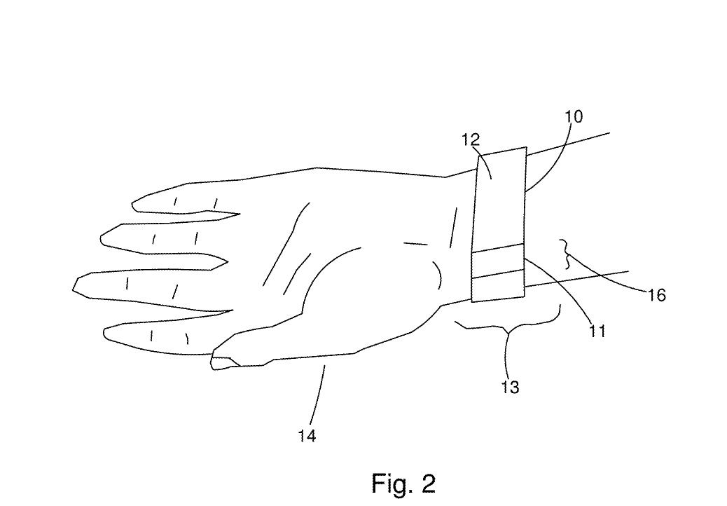

An exemplary wearable health-monitoring device 10 of the present invention is shown in FIG. 1. As shown in FIG. 1, exemplary wearable health-monitoring device 10 comprises a substrate 12 and a sensor 11 positioned along substrate 12. In this embodiment, exemplary substrate 12 of wearable health-monitoring device 10 comprises a wrist band with corresponding interlocking/latching fasteners 15 at opposite ends of substrate 12. FIG. 2 depicts exemplary wearable health-monitoring device 10 on patient 14 at a wrist location 13 of patient 14 so that sensor 11 is positioned at a pulse-taking location 16 of wrist location 13.

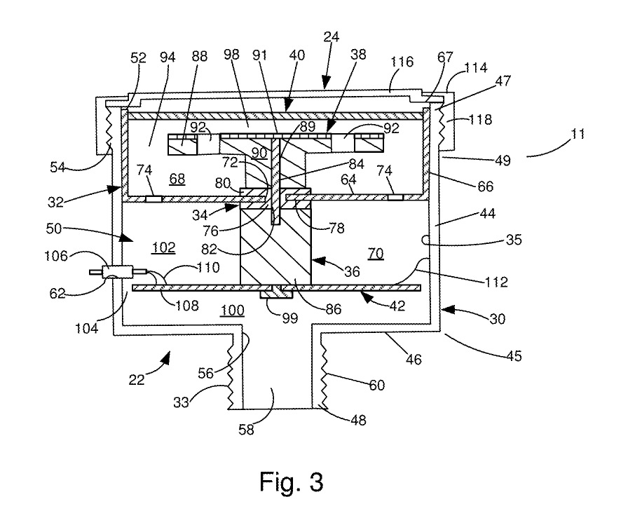

Sensor 11 may comprise a sensor similar to or identical to the sensor described in U.S. Patent Application Publication Number 2016/0095571, now U.S. Pat. No. 9,445,779, and shown in FIG. 3. Exemplary sensor 11 may comprise microphone 22, a cup-like body 30, a cup-like support plate 32, an insulating member 34, a conductor 36, a backplate 38, a membrane 40 and a low-noise preamplifier board 42. Body 30 has a cylindrical side wall 44 having a proximal end 45 and a distal end 47, an end wall 46 at proximal end 45 of body 30, and a connection port 48 extending proximally from end wall 46. Body 30 may be formed of metal, such as a stainless steel or aluminum. Side wall 44 and end wall 46 define an internal cavity 50 within body 30. Distal end 47 of body 30 is open such that an aperture 52 is defined in body 30. A thread form 54 is provided on the exterior surface 49 of side wall 44 at distal end 47.

End wall 46 substantially closes proximal end 45 of body 30 with the exception of an aperture 56 therethrough, and may extend perpendicularly relative to side wall 44. Aperture 56 may be centrally located in end wall 46 and is within connection port 48. Connection port 48 extends proximally from end wall 46 and has a passageway 58 therethrough, which is in communication with cavity 50 via aperture 56. Exterior surface 33 of connection port 48 has a thread form 60 thereon. An aperture 62 is provided through side wall 44 at a position spaced from proximal end 45 of side wall 44.

Support plate 32 is attached to an internal surface 35 of side wall 44 and seats within cavity 50. Support plate 32 may be formed of metal, and has a circular base wall 64, which spans the diameter of side wall 44 and is parallel to end wall 46, and a depending side wall 66, which extends distally from base wall 64. Side wall 66 terminates in a free end 67. Side wall 66 engages against internal surface 35 of side wall 44 of body 30, such that free end 67 of side wall 66 is proximate to distal end 47 of body 30, and base wall 64 is spaced from distal end 47 of body 30. Support plate 32 is affixed to body 30 by suitable means, such as welding, in such a way that whole assembly can be connected to the ground of preamplifier board 42. As a result of this arrangement, a distal chamber 68 is formed between base wall 64 and distal end 47 of body 30, and a proximal chamber 70 is formed between base wall 64 and proximal end 45 of body 30. Base wall 64 has an aperture 72 therethrough, which may be centrally located. Base wall 64 also has at least one aperture 74 or slot therethrough to allow air to flow from distal chamber 68 to proximal chamber 70.

The insulating member 34, which may be formed of plastic, ceramic, wood or any suitable insulating material, seats within aperture 72 in support plate 32 and is used to electrically isolate conductor 36, backplate 38 and preamplifier board 42 from support plate 32. As shown, insulating member 34 has a central portion 76, which extends through aperture 72, a proximal portion 78, which extends radially outwardly from central portion 76 on the distal side of base wall 64, and a distal portion 80, which extends radially outwardly from central portion 76 on the proximal side of base wall 64. A passageway 82 extends through central portion 76.

Backplate 38 is formed of a conducting material, and is formed from a base wall 88 and may further be formed of a proximal extending portion 90, which extends perpendicularly from base wall 88. Backplate 38 may be formed of, for example, from conducting ceramics, brass, or stainless steel. A passageway 89 extends through base wall 88, and extending portion 90 if provided, from its proximal surface to its distal surface. A permanently polarized thin polymer film 91 is coated on the distal surface of backplate 38. Polarized thin polymer film 91 operates without the need for an external power supply. As described in U.S. Pat. No. 8,401,217, the subject matter of which is hereby incorporated by reference in its entirety, backplate 38 has a plurality of spaced apart holes 92 therethrough (two holes are visible in FIG. 3). Extending portion 90 engages against distal portion 80 of insulating member 34, and is secured to a distal end of conductor 36, such that backplate 38 and conductor 36 are in electrical communication. Base wall 88 of backplate 38 is parallel to base wall 64 of support plate 32. A slot 94 is defined between the outer diameter of backplate 38 and side wall 44 of body 30. The area between backplate 38 and the proximal end 45 of body 30 defines a backchamber.

Conductor 36 extends through passageways 82, 89 and extends into proximal chamber 70. Conductor 36 is electrically connected to backplate 38. As shown, conductor 36 is formed of a conducting rod or wire 84, which extends through passageways 82, 89, and a conductive rod 86 extending proximally from conducting rod or wire 84 and insulating member 34. If formed of two components, the components are suitably connected to each other to form an electrical connection. Rod or wire 84 and rod 86 may be formed of brass, or may be formed of differing conductive materials. The proximal end of conductor 46 is proximate, to but spaced from, end wall 46 such that a gap is defined therebetween.

Membrane 40 is formed of a flexible conductive material and is seated at distal free end 67 of side wall 66 of support plate 32 such that the membrane 40 is positioned within distal chamber 68 and is proximate to, but spaced from, distal end 47 of body 30. The diameter of membrane 40 is selected so that membrane 40 stays within side wall 66. Membrane 40 is parallel to end wall 46 of body 30 and to base wall 64 of support plate 32. As a result, membrane 40 is in electrical communication with support plate 32. The tension of membrane 40 may be less than about 400 Newton per meter.

Backplate 38 is proximate to, but spaced from membrane 40, such that an air gap 98 is formed between membrane 40 and backplate 38 to create a capacitor in microphone 22 as is described in U.S. Pat. No. 8,401,217. As described in U.S. Pat. No. 8,401,217, the number, locations and sizes of holes 92, the size of slot 94, and the inner volume of the backchamber are selected such to allow enough air flow to provide proper damping of the motion of membrane 40. As described in U.S. Pat. No. 8,401,217, the backchamber serves as a reservoir for the airflow through holes 92 in backplate 38.

As described in U.S. Patent Application Publication Number 2016/0095571, now U.S. Pat. No. 9,445,779, in an exemplary embodiment, membrane 40 has a diameter of approximately 1.05 inches (0.0268 meter). Membrane 40 may have the following characteristics/dimensions: radius=0.0134 meter; thickness=2.54×10⁻⁵ meter; density=8000 kilogram/meter³; tension=400 N/meter; surface density=0.1780 kilogram/meter²; and stress=47.4045 PSI. Further, microphone 22 may comprises an air layer, which may have the following characteristics/dimensions: air gap=2.54×10⁻⁵ meter; density=1.2050 kilogram/meter³; viscosity=1.8×10⁻⁵ Pascal-second; sound velocity through the air gap=290.2 meters per second; and gamma=1.4. Microphone 22 may also comprise a slot 94, which may have the following characteristics/dimensions: distance from the center of the backplate=0.0117 meter; width=0.00351 meter; depth=0.00114 meter; and area=0.000258 meter². Backplate 38 may define six holes 92, and each hole 92 may have the following characteristics/dimensions: distance from center of backplate to center of hole=0.00526 meter; radius=0.002 meter; depth=0.045 meter; angle between two lines going from center of backplate to either side edge of hole=43.5 degrees; and area=1.26×10⁻⁵ meter². Microphone 22 may also have the following further characteristics/dimensions: volume of the backchamber=5×10⁻⁵ meter³; membrane mass=480 kilogram/meter²; membrane compliance=3.2×10⁻¹¹ meter⁵/Newton; and air gap compliance=3.5×10⁻¹⁰ meter⁵/Newton. In one exemplary embodiment, the resonant frequency of microphone 22 may be 3108.01 Hertz.

Preamplifier board 42 is planar and extends radially outwardly from the proximal end of conductor 36. Preamplifier board 42 is connected to the proximal end of conductor 36 by suitable means such that there is an electrical connection between preamplifier board 42 and conductor 36, such as a brass screw 99. Preamplifier board 42 is parallel to end wall 36 of body 30, base wall 64 of support plate 32 and base wall 88 of backplate 38. The position of preamplifier board 42 defines a first proximal chamber 100, which has a volume V1 between preamplifier board 42 and end wall 46 of body 30, and a second distal chamber 102, which has a volume V2 between preamplifier board 42 and base wall 64 of support plate 32. A slot 104 is defined between the outer diameter of preamplifier board 42 and side wall 44 of body 30 to allow air to flow from distal chamber 102 to proximal chamber 100. In one embodiment, volume V1 is approximately 0.1287 cubic inch, and volume V2 is approximately 0.6 cubic inch. The air can only flow from distal chamber 102 to proximal chamber 100 through slot 104. In one embodiment, slot 104 has a clearance distance between the outer diameter of preamplifier board 42 and side wall 44 of approximately 0.025″, which slot 104 extends around preamplifier board 42.

An electrical connection 106 extends through aperture 62 in side wall 44 and is sealed to side wall 44 by suitable means. Electrical connection 106 is electrical communication with preamplifier board 42 via wires 108, 110. Preamplifier board 42 is also electrically connected to body 30 via a wire 110, which provides a ground to preamplifier board 42. Preamplifier board 42 contains known components for measuring the capacitance between membrane 40 and backplate 38, and converting this measured capacitance into voltage.

Connection port 48 may be connected to a distal end of a flexible tube (i.e., such as flexible tube 26 shown in U.S. Patent Application Publication Number 2016/0095571, now U.S. Pat. No. 9,445,779), which may be formed of latex or rubber, and which has an earpiece (i.e., such as earpiece 28 shown in U.S. Patent Application Publication Number 2016/0095571, now U.S. Pat. No. 9,445,779) at the proximal end of the tube. Such a flexible tube and earpiece, like a typical stethoscope, are known in art for transmitting sound. The flexible tube, when present, is attached to connection port 48, such that there is no air exchange between the flexible tube and body 30, and such that the passageway through the tube is in communication with distal chamber 100 via passageway 58 and aperture 56. When the earpiece is inserted into the ears of the medical personnel, this allows substantially no air exchange between cavity 50 of microphone 22 and the outside of microphone 22. The length of the flexible tube is adjusted so that maximum audible sound is received at the earpiece, which are used by medical personnel to hear the desired sounds in real time.

In other embodiments, a cap (not shown) may be positioned over connection port 48 to seal this opening of body 30. In yet another embodiment, connection port 48 is not present, and end wall 46 of body 30 is continuous (i.e., there are no apertures/opening within or thru end wall 46).

The combination of volumes V1 and V2 and slot 104 around preamplifier board 42 provide sufficient acoustic resistance for pressure equalization, and lowers the low frequency threshold. When a flexible tube is connected to an earpiece, due to increased acoustic resistance and longer required period for pressure equalization, this lowers the low −3 dB frequency to 0.03 Hertz.

As described in U.S. Patent Application Publication Number 2016/0095571, now U.S. Pat. No. 9,445,779, the microphone may differ from U.S. Pat. No. 8,401,217 in that preamplifier board 42 is mounted horizontally in body 30 to divide the backchamber into two lower chambers 100 and 102 and that preamplifier board 42 is parallel to membrane 40, rather than being positioned vertically that is perpendicular to membrane 40 as is positioned in U.S. Pat. No. 8,401,217, and in that the grid of U.S. Pat. No. 8,401,217 is eliminated and instead body 30 includes threads 54 for connection of body coupler 24 (or body coupler 24a as discussed in U.S. Patent Application Publication Number 2016/0095571, now U.S. Pat. No. 9,445,779) to distal end 47 of body 30.

Body coupler 24 (or body coupler 24a) threadedly attaches to thread form 54 at distal end 47 of body 30 such that there is no air exchange between body coupler 24 (or body coupler 24a) and body 30. In one embodiment, as shown in FIG. 2, body coupler 24 is formed of an outer ring 114, which has a flexible non-conductive diaphragm 116 attached thereto and which spans the diameter of ring 114. Outer ring 114 may be formed either of thermoplastic polyurethane elastomers (TPU) or of closed cell polyurethane foam material, which can be made of different densities, and has an internal thread form 118 for attachment of outer ring 114 to distal end 47 of body 30. The TPU material is used when full spectrum of acoustic signals are to be recorded from a heart and closed cell polyurethane foam material is used only when infrasonic signals is to be recorded as this material acts as a passive filter and audible sound is shunted off. When attached, membrane 40 of microphone 22 and diaphragm 116 of body coupler 24 (or body coupler 24a) is approximately 0.1 inch apart. Body coupler 24 (or body coupler 24a) is placed against the body of the patient during the monitoring of the physiological process.

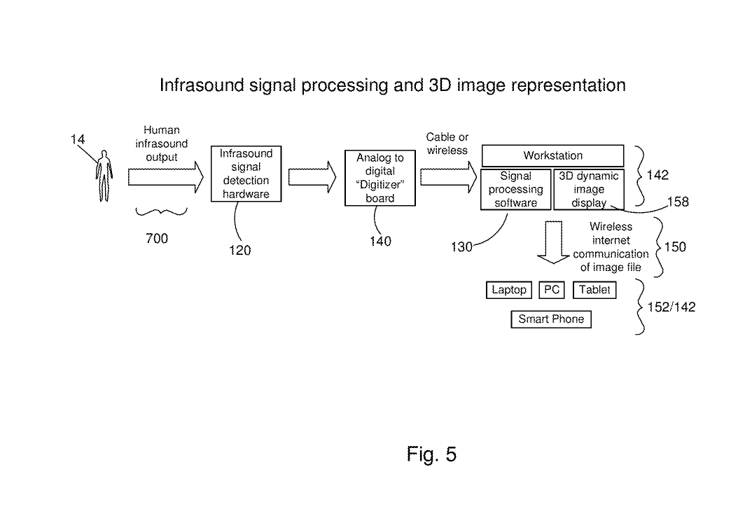

In some embodiments, a body coupler 24a as shown in FIG. 5A of U.S. Patent Application Publication Number 2016/0095571, now U.S. Pat. No. 9,445,779, may be used in sound detecting systems of the present invention and the sensor 11 used therein. However, in preferred embodiments of the present invention, a body coupler such as body coupler 24 is used in a non-invasive method of detecting infrasound of a patient (e.g., patient 14 shown in FIG. 2).

As discussed herein, preamplifier board 42 is installed parallel to base wall 54 and to membrane 24. Slot 104 between the edge of preamplifier board 42 and side wall 44 is small, for example 0.025″, to increase acoustic resistance. The combined volumes V1 and V2 and the volume in the flexible tube, when present, is less than or equal to about 5×10⁻⁵ meter³. Because of increased acoustic resistance, pressure equalization takes longer, which aids in lower −3 dB frequency to 0.03 Hertz.

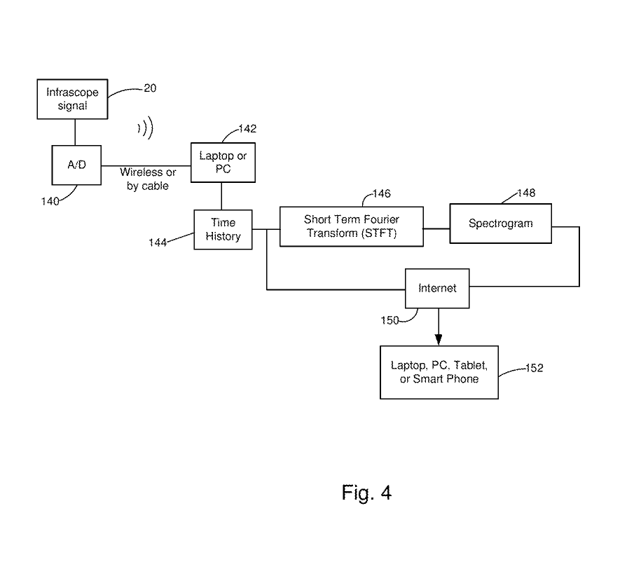

As shown in the block diagram of FIG. 4, in some embodiments, signals from sensor 11 may be digitized via an analog to digital digitizer board 140. Once digitized, the signal is transmitted wirelessly or by cable to workstation 142, such as a laptop or personal computer. At 144, time history is plotted for data collected at wrist location 13 of patient 14 as shown in FIG. 2. Workstation 142 provides control, analysis and display of the recorded data. MATLAB software may also be used to process the data to generate real-time spectrograms using short-time Fourier transform (STFT) spectrum of the corresponding data at 146 and 148. The time history and spectrogram of biological signals is transferred by the Internet 150 to a remote workstation 152, if desired, for observation and analysis. Examples of such remote workstations 152 may be a remote computer monitor, smartphone or tablet. The signals may be sent via wired connection, or may be wirelessly transmitted, such as by using commercially available Bluetooth module, to PC or laptop for processing. The data is converted in useful visual format also called spectrogram, which may be helpful for physician to diagnose any abnormality. The display of short term spectra is performed in real time, in order to detect the presence of a short term event in the data.

As shown in the block diagram of FIG. 5, in some embodiments, signals 700 from sensor 11 may be (i) detected using infrasound signal detection hardware 120 (e.g., the device as described in U.S. Pat. No. 8,401,217, namely, a sensor and integrated pre-amplification board), (ii) digitized via an analog to digital digitizer board 140, and (iii) transmitted wirelessly or by cable to one or more workstations 142, such as a laptop or personal computer, and (iv) converted into one or more files (e.g., text and/or image files). The one or more files may be subsequently transmitted wirelessly or by cable to one or more workstations 142 and/or one or more remote workstations 152, if desired, for observation and analysis. In some embodiments, the step of converting signals 700 from sensor 11 into one or more files may be performed via signal processing software 130 (e.g., any number of commercially available three-dimensional image processing software packages) so as to generate three dimensional (3D) images, which may be displayed on a 3D dynamic image display 158.

The wearable health-monitoring devices and methods of the present invention are further described in the following embodiments.

### Other Embodiments

Wearable Health-Monitoring Devices

1. A wearable health-monitoring device comprising: (1) a substrate that is attachable to a patient's body; and (2) one or more sensors attached to or embedded within the substrate, wherein each sensor comprises: a body comprising a proximal end, a distal end, a body side wall extending between the proximal end and the distal end, an end wall at the proximal end, and an aperture at the distal end; a body coupler attached to the distal end and over the aperture so as to form a substantially air-tight seal, wherein the body coupler is capable of engagement with the patient; a cavity surrounded by the body side wall, the end wall and the body coupler; a conductive backplate within the cavity and defining a backchamber between the conductive backplate and the end wall; a conductive membrane within the cavity, the conductive backplate and the conductive membrane being spaced apart from each other to form a capacitor; and a preamplifier board in electrical connection with the conductive backplate, the preamplifier (i) being capable of measuring a capacitance between the conductive membrane and the conductive backplate and converting the measured capacitance into a voltage signal, and (ii) being parallel to each of the conductive backplate and the conductive membrane, each of said one or more sensors being capable of detecting acoustic signals in a frequency range of 0.01 Hertz (Hz) to 30 Hz (or any value between 0.01 Hz and 30 Hz including end points 0.01 Hz and 30 Hz, in increments of 0.01 Hz, e.g., 0.05 Hz, or any range of value between 0.01 Hz and 30 Hz including end points 0.01 Hz and 30 Hz, in increments of 0.01 Hz, e.g., from 0.81 Hz to 8.75 Hz). In some embodiments, each sensor is the sensor described in International Patent Application No. PCT/US2015/020964, filed on 17 Mar. 2015, which claims the benefit of and priority to U.S. Non-Provisional patent application Ser. No. 14/658,584, filed on Mar. 16, 2015, which claims the benefit of and priority to U.S. Provisional Patent Application Ser. No. 62/058,794, filed on Oct. 2, 2014, the contents of all of which are incorporated by reference herein in their entirety. In other embodiments, each sensor may be a slightly modified version of the sensor described in International Patent Application No. PCT/US2015/020964. As used herein, the term “attachable” refers to a substrate that is (a) actually physically attached to a patient's body via a material such as an adhesive, or (b) positionable on, but not physically attached to, the skin of a patient's body via an adhesive (e.g., a temporary bandage or wristband containing the sensor) or a mechanical device (e.g., hook and loop material used on a wristband, a zipper used on a piece of clothing that positions the sensor next to the skin on the patient's body, etc.) or (c) positionable proximate to, but not on the skin or physically attached to the skin of, a patient's body via any adhesive (e.g., a temporary bandage or piece of clothing that positions the sensor over a patient's clothing) or a mechanical device (e.g., hook and loop material used on a wristband, a zipper used on a piece of clothing that positions the sensor over a patient's clothing, etc.).
2. The wearable health-monitoring device of embodiment 1, wherein each sensor further comprises: a conductive support plate attached to an internal surface of the body side wall within the cavity, the conductive support plate (i) comprising a base wall that divides the cavity into a distal chamber between the base wall and the distal end of the body and a proximal chamber between the base wall and the proximal end of the body, (ii) a base wall aperture within the base wall, and (iii) at least one aperture or slot within the base wall to allow air to flow from the distal chamber to the proximal chamber; an insulating member extending through the base wall aperture in the conductive support plate; a conductor extending through the insulating member and extending therefrom, the conductive member being electrically connected to the conductive backplate and to the preamplifier board, wherein the conductive backplate is on one side of the conductive support plate and the preamplifier board is on an opposite side of the conductive support plate.
3. The wearable health-monitoring device of embodiment 1 or 2, wherein (i) the conductive backplate defines a plurality of holes, (ii) a slot is defined between an outer diameter of the conductive backplate and an inner wall of the body, and (iii) locations and sizes of the holes and a size of the slot are selected such that membrane motion is substantially critically damped.
4. The wearable health-monitoring device of any one of embodiments 1 to 3, wherein the conductive backplate is seated on the insulating member.
5. The wearable health-monitoring device of any one of embodiments 1 to 4, wherein a slot is defined between the preamplifier board and the body side wall, and extends around the preamplifier board.
6. The wearable health-monitoring device of any one of embodiments 1 to 5, wherein the preamplifier board defines a first proximal chamber between the preamplifier board and the end wall, and a second distal chamber between the preamplifier board and the base wall of the conductive support plate.
7. The wearable health-monitoring device of embodiment 6, wherein the first proximal chamber has a volume of approximately 0.1287 cubic inch, and the second distal chamber has a volume of approximately 0.6 cubic inch.
8. The wearable health-monitoring device of any one of embodiments 1 to 7, wherein the body coupler is formed of an outer ring having a flexible, non-conductive diaphragm attached thereto, and the outer ring is attached to the body.
9. The wearable health-monitoring device of any one of embodiments 1 to 8, further comprising a sealed electrical connection extending though the body side wall, said sealed electrical connection enabling electrical connection of said one or more sensors to an electronics board.
10. The wearable health-monitoring device of any one of embodiments 1 to 9, further comprising a digitizer board which is remote from the sensor, said digitizer board being capable of digitizing the voltage signal from the preamplifier.
11. The wearable health-monitoring device of any one of embodiments 1 to 10, wherein the voltage signal is digitized and electronically transmitted to a remote location.
12. The wearable health-monitoring device of any one of embodiments 1 to 11, wherein each of said one or more sensors is capable of detecting all acoustic signals having a frequency of from 0.01 Hz to 30 Hz in increments of 0.01 Hz.
13. The wearable health-monitoring device of any one of embodiments 1 to 12, wherein said device is capable of providing direct feedback to the patient if an adverse health condition is detected by the sensor.
14. The wearable health-monitoring device of any one of embodiments 1 to 13, wherein said device is capable of providing direct feedback to the patient if an adverse health condition is detected by the one or more sensors, the direct feedback comprising a visual signal, an audio signal, a vibrational signal, or any combination thereof. An example of direct feedback would be a visual, audio, and/or vibrational such as “Contact your general practitioner for follow-up” or “Call 911 now.”

15. The wearable health-monitoring device of any one of embodiments 1 to 14, wherein said device is capable of communicating patient infrasound data directly to the patient's general practitioner or any other healthcare professional via an Internet message for their immediate review.

16. The wearable health-monitoring device of any one of embodiments 1 to 15, wherein said device is capable of providing a cardiovascular disease score from 1-100 to enable the patient to understand their cardiovascular health.
17. The wearable health-monitoring device of any one of embodiments 1 to 16, wherein the substrate comprises a wrist band sized to fit around the patient's wrist.
18. The wearable health-monitoring device of any one of embodiments 1 to 17, wherein the substrate comprises a wrist band sized to fit around the patient's wrist, and when positioned around the patient's wrist, the substrate positions said one or more sensors at a pulse-taking location on the patent's wrist (i.e., a location on an underside of the patient's wrist where a pulse is typically taken). Although not shown in the figures, the substrate may further comprise one or more additional features such as an actual watch, a second sensor for detecting other physiological properties of a patient, exercise monitoring features (e.g., counting steps, distance of walking/running, heartrate, etc.).
19. The wearable health-monitoring device of any one of embodiments 1 to 16, wherein the substrate comprises a piece of clothing sized to fit around any portion of the patient's body. Suitable pieces of clothing include, but are not limited to, a headband, a vest, a sock, a bandanna, a shirt, a pair of pants, a gown, an undergarment, stockings, a coat/jacket, etc. Any of the above-mentioned pieces of clothing may be attached and/or positioned on a patient via any of the above-described adhesives, mechanical devices, etc. In some cases, such as socks, elastic material within the socks provides enough of an attachment force to hold a sensor in place next to or on the patient's body.
20. The wearable health-monitoring device of any one of embodiments 1 to 19, wherein each sensor has a rectangular shape and a length of about 1 inch and a width of from about ¼ inch to about ½ inch.
21. The wearable health-monitoring device of any one of embodiments 17 to 18 and 20, wherein the wrist band comprises a strap of material and engaging fasteners on opposite ends of the strap of material. The strap of material may comprise any material including, but not limited to, leather, plastic, fabric, metal, or any combination thereof.
22. The wearable health-monitoring device of any one of embodiments 1 to 21, wherein the one or more sensors comprises two or more sensors, and each sensor comprises the sensor described in any one of embodiments 1-16 and 20. For example, in some embodiments, the wearable health-monitoring device may comprise a vest (or other piece of clothing) with multiple sensors positioned at multiple locations along the vest (or other piece of clothing) so as to be able to measure infrasonic activity at multiple locations along the patient's body. It should be further understood that the wearable health-monitoring device of any one of embodiments 1 to 21 may further comprise one or more sensors other than the sensors described in any one of embodiments 1-16 and 20. For example, one or more second sensors that detect and process an EKG output (or other types of non-infrasound sensors) may be used in combination with the “infrasound-type” sensors in the wearable health-monitoring devices of the present invention.

Methods of Making Wearable Health-Monitoring Devices

23. A method of making the wearable health-monitoring device of any one of embodiments 1 to 22, said method comprising: attaching or embedding the sensor onto or in the substrate.

Methods of Using Wearable Health-Monitoring Devices

24. A method of using the wearable health-monitoring device of any one of embodiments 1 to 22, said method comprising: positioning the wearable health-monitoring device so that the one or more sensors can detect sound from one or more locations within the patient's body.
25. The method of embodiment 24, wherein the wearable health-monitoring device is positioned on the patient's body so that at least one sensor within the one or more sensors is located at a pulse-taking location on the patent's wrist (i.e., a location on an underside of the patient's wrist where a pulse is typically taken).
26. The method of embodiment 24, wherein the wearable health-monitoring device is positioned on the patient's body so that at least one sensor within the one or more sensors is located at a location on the patient's body other than a patient's wrist (e.g., along the patient's chest, the patient's temple, the patient's neck region, etc.).
27. The method of any one of embodiments 24 to 26, wherein the wearable health-monitoring device is positioned on the patient's body so that two or more sensors within the one or more sensors are located at two or more different locations on the patient's body (e.g., along the patient's wrist, the patient's chest, the patient's temple, the patient's neck region, etc., or any combination thereof).

It should be understood that although the above-described wearable health-monitoring devices, and methods are described as “comprising” one or more components or steps, the above-described compositions, and methods may “comprise,” “consists of” or “consist essentially of” any of the above-described components or steps of the compositions, and methods. Consequently, where the present invention, or a portion thereof, has been described with an open-ended term such as “comprising,” it should be readily understood that (unless otherwise stated) the description of the present invention, or the portion thereof, should also be interpreted to describe the present invention, or a portion thereof, using the terms “consisting essentially of” or “consisting of” or variations thereof as discussed below.

As used herein, the terms “comprises,” “comprising,” “includes,” “including,” “has,” “having,” “contains”, “containing,” “characterized by” or any other variation thereof, are intended to encompass a non-exclusive inclusion, subject to any limitation explicitly indicated otherwise, of the recited components. For example, a wearable health-monitoring device and/or method that “comprises” a list of elements (e.g., components or steps) is not necessarily limited to only those elements (or components or steps), but may include other elements (or components or steps) not expressly listed or inherent to the wearable health-monitoring device and/or method.

As used herein, the transitional phrases “consists of” and “consisting of” exclude any element, step, or component not specified. For example, “consists of” or “consisting of” used in a claim would limit the claim to the components, materials or steps specifically recited in the claim except for impurities ordinarily associated therewith (i.e., impurities within a given component). When the phrase “consists of” or “consisting of” appears in a clause of the body of a claim, rather than immediately following the preamble, the phrase “consists of” or “consisting of” limits only the elements (or components or steps) set forth in that clause; other elements (or components) are not excluded from the claim as a whole.

As used herein, the transitional phrases “consists essentially of” and “consisting essentially of” are used to define a wearable health-monitoring device and/or method that includes materials, steps, features, components, or elements, in addition to those literally disclosed, provided that these additional materials, steps, features, components, or elements do not materially affect the basic and novel characteristic(s) of the claimed invention. The term “consisting essentially of” occupies a middle ground between “comprising” and “consisting of”.

Further, it should be understood that the herein-described wearable health-monitoring devices, and methods may comprise, consist essentially of, or consist of any of the herein-described components and features, as shown in the figures with or without any feature(s) not shown in the figures. In other words, in some embodiments, the wearable health-monitoring devices and/or methods of the present invention do not have any additional features other than those shown in the figures, and such additional features, not shown in the figures, are specifically excluded from the wearable health-monitoring devices and/or methods. In other embodiments, the wearable health-monitoring devices and/or methods of the present invention do have one or more additional features that are not shown in the figures.

The present invention is further illustrated by the following examples, which are not to be construed in any way as imposing limitations upon the scope thereof. On the contrary, it is to be clearly understood that resort may be had to various other embodiments, modifications, and equivalents thereof which, after reading the description herein, may suggest themselves to those skilled in the art without departing from the spirit of the present invention and/or the scope of the appended claims.

### Example 1

Wearable health-monitoring devices as described in embodiments 1 to 22 and as shown in the figures were prepared.

While the specification has been described in detail with respect to specific embodiments thereof, it will be appreciated that those skilled in the art, upon attaining an understanding of the foregoing, may readily conceive of alterations to, variations of, and equivalents to these embodiments. Accordingly, the scope of the present invention should be assessed as that of the appended claims and any equivalents thereto.

What is claimed is:

1. A wearable health-monitoring device comprising:
a substrate that is attachable to a patient's body; and
one or more sensors attached to or embedded within the substrate, wherein each of the one or more sensors comprises:
a body comprising a proximal end, a distal end, a body side wall extending between the proximal end and the distal end, an end wall at the proximal end, and an aperture at the distal end;
a body coupler attached to the distal end and over the aperture so as to form a substantially air-tight seal, wherein the body coupler is capable of engagement with the patient;
a cavity surrounded by the body side wall, the end wall and the body coupler;
a conductive backplate within the cavity and defining a backchamber between the conductive backplate and the end wall;
a conductive membrane within the cavity, the conductive backplate and the conductive membrane being spaced apart from each other to form a capacitor; and
a preamplifier board in electrical connection with the conductive backplate, the preamplifier (i) being capable of measuring a capacitance between the conductive membrane and the conductive backplate and converting the measured capacitance into a voltage signal, and (ii) being parallel to each of the conductive backplate and the conductive membrane,
each of said one or more sensors being capable of detecting acoustic signals in a frequency range of 0.01 Hertz (Hz) to 30 Hz.

2. The wearable health-monitoring device of claim 1, wherein each of said one or more sensors further comprises:
a conductive support plate attached to an internal surface of the body side wall within the cavity, the conductive support plate (i) comprising a base wall that divides the cavity into a distal chamber between the base wall and the distal end of the body and a proximal chamber between the base wall and the proximal end of the body, (ii) a base wall aperture within the base wall, and (iii) at least one aperture or slot within the base wall to allow air to flow from the distal chamber to the proximal chamber;
an insulating member extending through the base wall aperture in the conductive support plate;
a conductor extending through the insulating member and extending therefrom, the conductive member being electrically connected to the conductive backplate and to the preamplifier board,
wherein the conductive backplate is on one side of the conductive support plate and the preamplifier board is on an opposite side of the conductive support plate.

3. The wearable health-monitoring device of claim 2, wherein (i) the conductive backplate defines a plurality of holes, (ii) a slot is defined between an outer diameter of the conductive backplate and an inner wall of the body, and (iii) locations and sizes of the holes and a size of the slot are selected such that membrane motion is substantially critically damped.

4. The wearable health-monitoring device of claim 1, wherein the conductive backplate is seated on the insulating member.

5. The wearable health-monitoring device of claim 1, wherein a slot is defined between the preamplifier board and the body side wall, and extends around the preamplifier board.

6. The wearable health-monitoring device of claim 1, wherein the preamplifier board defines a first proximal chamber between the preamplifier board and the end wall, and a second distal chamber between the preamplifier board and the base wall of the conductive support plate.

7. The wearable health-monitoring device of claim 6, wherein the first proximal chamber has a volume of approximately 0.1287 cubic inch, and the second distal chamber has a volume of approximately 0.6 cubic inch.

8. The wearable health-monitoring device of claim 1, wherein the body coupler is formed of an outer ring having a flexible, non-conductive diaphragm attached thereto, and the outer ring is attached to the body.

9. The wearable health-monitoring device of claim 1, further comprising a sealed electrical connection extending though the body side wall, said sealed electrical connection enabling electrical connection of said one or more sensors to an electronics board.

10. The wearable health-monitoring device of claim 1, further comprising a digitizer board which is remote from the one or more sensors, said digitizer board being capable of digitizing the voltage signal from the preamplifier.

11. The wearable health-monitoring device of claim 1, wherein the voltage signal is digitized and electronically transmitted to a remote location.

12. The wearable health-monitoring device of claim 1, wherein each of said one or more sensors is capable of detecting all acoustic signals having a frequency of from 0.01 Hz to 30 Hz in increments of 0.01 Hz.

13. The wearable health-monitoring device of claim 1, wherein said device is capable of providing direct feedback to the patient if an adverse health condition is detected by the one or more sensors.

14. The wearable health-monitoring device of claim 1, wherein said device is capable of providing direct feedback to the patient if an adverse health condition is detected by the sensor, the direct feedback comprising a visual signal, an audio signal, a vibrational signal, or any combination thereof.

15. The wearable health-monitoring device of claim 1, wherein said device is capable of communicating patient infrasound data directly to the patient's general practitioner or any other healthcare professional via an Internet message for their immediate review.

16. The wearable health-monitoring device of claim 1, wherein the substrate comprises a wrist band sized to fit around the patient's wrist.

17. The wearable health-monitoring device of claim 1, wherein the substrate comprises a wrist band sized to fit around the patient's wrist, and when positioned around the patient's wrist, the substrate positions said sensor at a pulse-taking location on the patent's wrist.

18. The wearable health-monitoring device of claim 16, wherein each of said one or more sensors has a rectangular shape and a length of about 1 inch and a width of from about ¼ inch to about ½ inch, and the wrist band comprises a strap of material and engaging fasteners on opposite ends of the strap of material.

19. A method of making the wearable health-monitoring device of claim 1, said method comprising:
attaching or embedding the one or more sensors onto or in the substrate.

20. A method of using the wearable health-monitoring device of claim 1, said method comprising:
positioning the wearable health-monitoring device so that the one or more sensors can detect sound from one or more locations within the patient's body.
